(19) United States
(12) Patent Application Publication
(10) Pub. No.: US 2026/0151073 A1
(43) Pub. Date: Jun. 4, 2026

(54) METHOD FOR DETERMINING CARDIAC CONDUCTION VELOCITY AND DEVICE FOR DETERMINING CARDIAC CONDUCTION VELOCITY USING THE SAME

(71) Applicant: INDUSTRY-ACADEMIC COOPERATION FOUNDATION, YONSEI UNIVERSITY, Seoul (KR)

(72) Inventors: Hui-Nam PAK, Seoul (KR); Oh-Seok KWON, Goyang-si (KR)

(21) Appl. No.: 19/403,467

(22) Filed: Nov. 28, 2025

(30) Foreign Application Priority Data
Nov. 29, 2024 (KR) 10-2024-0175784

(51) Int. Cl. A61B 5/367; A61B 5/00

(52) U.S. Cl. CPC A61B 5/367; A61B 5/0044; A61B 5/7275; A61B 5/7435; A61B 5/749; A61B 2576/023

(57) ABSTRACT

The present invention provides a method for determining a cardiac conduction velocity, a device using the same, a cardiac 3D structure imaging device and a system for determining a cardiac conduction velocity, and the method is implemented by a processor, comprising receiving heart 3D data including vertex coordinates for a plurality of triangles constituting a heart structure, calculating a normal vector for a target triangle based on the vertex coordinates of the target triangle selected among the plurality of triangles, determining the cardiac conduction velocity for the target triangle based on the normal vector, and determining the cardiac conduction velocity for the target vertex based on a cardiac conduction velocity for an adjacent triangle including the target vertex selected among the vertices of the target triangle among the plurality of triangles.

### CROSS-REFERENCE TO RELATED APPLICATIONS

This application claims the priority of Korean Patent Application No. 10-2024-0175784 filed on Nov. 29, 2024, in the Ministry of Intellectual Property (MOIP) of Korea, the disclosure of which is incorporated herein by reference.

### BACKGROUND

### Field

The present invention relates to a method for determining cardiac conduction velocity and a device for determining cardiac conduction velocity using the same.

### Description of the Related Art

Cardiac conduction velocity (CV) refers to the rate at which electrical signals from the heart are transmitted through the myocardium and is considered an important index for assessing the electrophysiological state of the heart.

At this time, the CV quantitatively indicates the process of transmitting electric signals in the heart, and through this, the functional and physical states of the heart can be identified. More specifically, normal electrical signal transmission of the heart plays a key role in tuning the heart rate, and abnormalities in conduction velocity can be closely associated with heart disease.

In particular, the CV provides essential information in the diagnosis and analysis of pathological causes of various cardiovascular diseases, myocardial infarction, and heart failure as well as cardiac rhythm disorders such as arrhythmia.

For this reason, the technology to accurately measure and analyze the CV can be utilized as an important tool in medical diagnosis and treatment planning.

Accordingly, there is a continuous demand for the development of a technology capable of more accurately assessing the CV.

The background technology of the present invention was written to facilitate understanding of the present invention. It should not be understood that the matters described in the background technology of the invention exist as prior art.

### SUMMARY

On the other hand, the trigonometry, which is one of the methods for calculating the CV, is one of the methods for estimating the CV by calculating the gradient for each side of the triangle based on the local activation time (LAT).

However, the trigonometry may be limited in the calculation of the CV, especially in a 3D heart mesh structure, due to irregular shapes of triangles that form the basis of calculation, or depending on the propagation direction of the local activation time, i.e., the propagation direction and the array of the triangles in which the triangle is orthogonal.

As a result, the number of measurable triangles in the 3D heart mesh structure may decrease or may lead to distorted results of conduction velocity.

At this time, the electrical signal in the heart may have anisotropic properties, which appears in a complex form depending on the anatomical structure and electrical activity of the heart.

However, prior art techniques, including trigonometry, do not sufficiently reflect such anisotropic properties or complex structural elements, so there may be limitations in providing important data for diagnosis and treatment of heart disease.

In particular, as described above, the method for calculating cardiac conduction velocity using the trigonometry may pose a risk of producing unreliable results.

In order to overcome the limitations of the prior art described above, the inventors of the present invention have attempted to develop a novel method for calculating the CV.

As a result, the inventors of the present invention have come to develop a cardiac conduction velocity determining system capable of estimating a more accurate conduction velocity while preserving the topology of 3D data.

More specifically, the inventors of the present invention have introduced the calculation of the average nodal gradient in a novel system for determining cardiac conduction velocity. Through this, it was possible to develop a cardiac conduction velocity determining system that calculates a normal vector for a plane in each triangle, determines the gradient, and integrates the cardiac conduction velocity data of adjacent triangles to estimate the cardiac conduction velocity for a specific point.

Through this, the inventors of the present invention were able to recognize that the present invention can accurately calculate the cardiac conduction velocity even under conditions such as non-uniform size, ratio, and array of triangles.

Moreover, the inventors of the present invention were able to recognize that by providing a novel system for determining a cardiac conduction velocity it is possible to overcome the unmeasurable limitations caused by mismatch between the local activation time and the array of triangles in the prior art, and to provide consistent results even in complex structural environments which include both anisotropy and isotropy.

Furthermore, the inventors of the present invention were able to recognize that by providing a novel system for determining a cardiac conduction velocity it is possible to quantitatively analyze the transmission path of electrical signals in the heart through a novel approach based on gradient calculation methods, thereby contribute to more accurately diagnosing and effectively treating heart diseases such as arrhythmias.

Accordingly, an object to be achieved by the present invention is to provide a method for determining a cardiac conduction velocity based on an average nodal gradient calculation, which can reflect complex electrophysiologic characteristics of the heart, and a device using the same.

Objects of the present invention are not limited to the above-mentioned objects, and other objects, which are not mentioned above, can be clearly understood by those skilled in the art from the following descriptions.

In order to solve the above problems, a method for determining a cardiac conduction velocity according to an embodiment of the present invention is provided. The method is an information providing method implemented by a processor, comprising receiving heart 3D data including vertex coordinates for a plurality of triangles constituting a heart structure, calculating a normal vector for a target triangle based on the vertex coordinates of the target triangle selected among the plurality of triangles, determining a cardiac conduction velocity for the target triangle based on the normal vector, and determining a cardiac conduction velocity for the target vertex based on the cardiac conduction velocity for an adjacent triangle including the target vertex selected among the vertices of the target triangle among the plurality of triangles.

According to a feature of the present invention, the heart 3D data further includes a local activation time (LAT) corresponding to each vertex coordinate, and determining the cardiac conduction velocity for the target triangle may include calculating a gradient based on a normal vector and a local activation time value, and determining a cardiac conduction velocity for the target triangle based on the gradient.

According to another feature of the present invention, calculating the gradient may include calculating a difference in local activation time between vertices of a target triangle, calculating a difference in vector between vertices of the target triangle, calculating an area of the target triangle, and calculating a gradient based on the difference in local activation time, the difference in vector, the normal vector, and the area.

According to another feature of the present invention, the target triangle includes a first vertex, a second vertex, and a third vertex, and calculating a gradient may include calculating a first gradient, calculating a second gradient, and determining a gradient based on a sum of the first gradient and the second gradient. In this case, the first gradient may be defined as a gradient based on a normal vector and a difference in local activation time between the first vertex and the second vertex, and the second gradient may be defined as a gradient based on a normal vector and a difference in local activation time between the second vertex and the third vertex.

According to another feature of the present invention, determining the cardiac conduction velocity may include calculating a gradient for each of the adjacent triangles, determining a cardiac conduction velocity for each of the adjacent triangles based on the gradient, and calculating an average value of the cardiac conduction velocity for each of the adjacent triangles.

According to another feature of the present invention, the plurality of triangles may have a mesh structure corresponding to electrical signal conduction of the heart with respect to the heart surface.

According to another feature of the present invention, the plurality of triangles may be configured in an isotropic mesh or an anisotropic mesh structure according to conduction characteristics of heart tissue.

According to another feature of the present invention, the method may further comprise, after the determining the cardiac conduction velocity, determining that if the cardiac conduction velocity is below or above a predetermined level, the risk of developing a heart disease is high.

In order to solve the above-described problem, a device for determining a cardiac conduction velocity according to another embodiment of the present invention is provided. The device comprises a communication unit configured to receive heart 3D data comprising vertex coordinates for a plurality of triangles constituting a heart structure, and a processor functionally connected with the communication unit, wherein the processor is configured to calculate a normal vector for the target triangle based on the vertex coordinates of a target triangle selected among the plurality of triangles, determine a cardiac conduction velocity for the target triangle based on the normal vector, and determine a cardiac conduction velocity for the target vertex based on the cardiac conduction velocity for an adjacent triangle including the target vertex selected among the vertices of the target triangle among the plurality of triangles.

According to a feature of the present invention, the heart 3D data includes a local activation time (LAT) corresponding to each vertex coordinate, and the processor may be further configured to calculate a gradient based on the normal vector and the local activation time value, and determine the cardiac conduction velocity for the target triangle based on the gradient.

According to another feature of the present invention, the processor may be further configured to calculate a difference in local activation time between vertices of the target triangle, calculate a difference in vector between vertices of the target triangle, calculate an area of the target triangle, and calculate a gradient based on the difference in local activation time, the difference in vector, the normal vector, and the area.

According to another feature of the present invention, the target triangle includes a first vertex, a second vertex, and a third vertex, and the processor is further configured to calculate the first gradient, calculate the second gradient, and determine the gradient based on a sum of the first gradient and the second gradient, the first gradient being defined as a gradient based on a normal vector, and a difference in local activation time between the first vertex and the second vertex, and the second gradient being defined as a gradient based on as a normal vector and a difference in local activation time between the second vertex and the third vertex.

According to another feature of the present invention, the processor may be further configured to calculate a gradient for each of the adjacent triangles, determine a cardiac conduction velocity for each of the adjacent triangles based on the gradient, and calculate an average value of the cardiac conduction velocity for each of the adjacent triangles.

According to another feature of the present invention, the processor may be further configured to determine that if the cardiac conduction velocity is below or above a predetermined level, the risk of developing a heart disease is high.

In order to solve the above-described problem, a cardiac 3D structure imaging device according to another embodiment of the present invention is provided. The device comprises an imaging unit configured to capture a 3D structure of a heart, and generate heart 3D data consisting of a plurality of triangles and including the vertex coordinates of the plurality of triangles constituting the heart structure, a communication unit functionally connected to the imaging unit and configured to receive the heart 3D data, and a processor functionally connected to the communication unit or the imaging unit. In this case, the processor is configured to calculate a normal vector for a target triangle based on vertex coordinates of the target triangle selected among the plurality of triangles, determine a cardiac conduction velocity for the target triangle based on the normal vector, and determine a cardiac conduction velocity for the target vertex based on the cardiac conduction velocity for an adjacent triangle including the target vertex selected among the vertices of the target triangle among the plurality of triangles.

In order to solve the problems as described above, a system for determining a cardiac conduction velocity according to another embodiment of the present invention is provided. The system comprises an internal memory configured to store heart 3D data including vertex coordinates for a plurality of triangles constituting a heart structure, and is configured to calculate a normal vector for a target triangle based on the vertex coordinates of the target triangle selected among the plurality of triangles, determine a cardiac conduction velocity for the target triangle based on the normal vector, and determine a cardiac conduction velocity for the target vertex based on the cardiac conduction velocity for an adjacent triangle including the target vertex selected among the vertices of a target triangle among the plurality of triangles.

Other detailed matters of the embodiments are included in the detailed description and the drawings.

The present invention may provide a method for determining a cardiac conduction velocity capable of minimizing a deviation and improving the accuracy of a cardiac conduction velocity calculation by calculating a gradient of each triangle forming a heart structure and then integrating data of adjacent triangles by introducing a gradient calculation method. Accordingly, it is possible to secure more reliable data.

In particular, the present invention, may overcome the limitations of the prior art and derive results suitable for various clinical environments by providing a system capable of stably calculating a cardiac conduction velocity even in a 3D mesh environment where the size, ratio, array, etc. of triangles are irregular or uneven.

More specifically, the present invention can be applied to heart 3D data with various anatomical conditions and physical properties by providing stable and consistent results even in complex structural environments including both anisotropy and isotropy.

Furthermore, the present invention can contribute to the accurate diagnosis and treatment planning of cardiovascular diseases, including arrhythmia, by providing a system capable of quantitatively analyzing the transmission path of electrical signals in the heart.

That is, the present invention can provide highly accurate information on the cardiac conduction velocity, which is clinically important, thereby contributing to the development of personalized treatment strategies for patients with cardiovascular disease and the prediction of disease prognosis.

The effects according to the present invention are not limited to the contents exemplified above, and more various effects are included in the present invention.

### BRIEF DESCRIPTION OF DRAWINGS

FIG. 1 illustrates a system for determining a cardiac conduction velocity using a device for determining a cardiac conduction velocity according to an embodiment of the present invention.

FIG. 2A is a block diagram illustrating a configuration of a user device according to an embodiment of the present invention.

FIG. 2B is a block diagram showing the configuration of a decision server according to an embodiment of the present invention.

FIGS. 3A to 3D illustrate a procedure of a method for determining a cardiac conduction velocity according to an embodiment of the present invention.

FIGS. 4A to 4C exemplarily illustrate a procedure of a method for determining a cardiac conduction velocity according to an embodiment of the present invention.

FIGS. 5A to 5C show assessment results of a system for determining a cardiac conduction velocity according to various embodiments of the present invention.

### DETAILED DESCRIPTION OF THE EMBODIMENT

The advantages and features of the present invention, and the methods for achieving them, will become clear with reference to the embodiments described in detail below together with the attached drawings. However, the present invention is not limited to the embodiments disclosed below, but may be implemented in various different forms, and these embodiments are provided only to make the disclosure of the present invention complete and to fully inform a person having ordinary knowledge in the technical field to which the present invention belongs of the scope of the disclosure, and the present invention is defined only by the scope of the claims.

The shapes, sizes, ratios, angles, numbers, and the like illustrated in the accompanying drawings for describing the exemplary embodiments of the present invention are merely examples, and the present invention is not limited thereto. Further, in the following description of the present invention, a detailed explanation of known related technologies may be omitted to avoid unnecessarily obscuring the subject matter of the present invention. The terms such as “including,” “having,” and “consist of” used herein are generally intended to allow other components to be added unless the terms are used with the term “only”. When the component is expressed as a singular, it includes the case of including the plural unless otherwise explicitly stated.

Components are interpreted to include an error range even if there is no separate explicit description.

The features of various embodiments of the present invention can be partially or entirely coupled to or combined with each other and can be interlocked and operated in technically various ways, as can be fully understood by the person skilled in the art, and each embodiment can be carried out independently of or in association with each other.

For clarity of the interpretation of the present invention, terms used in the present invention will be defined below.

The term “heart 3D data” used in the present invention refers to data indicating a 3D structure of a heart and characteristics thereof.

According to an embodiment of the present invention, the heart 3D data may be a data composed of a plurality of triangles and having electrical conduction characteristics in a 3D mesh format.

In this case, the heart 3D data may include vertex coordinates and electrical properties of each triangle when the surface of the heart is modeled with a mesh structure composed of a plurality of triangles.

According to another embodiment of the present invention, the heart 3D data may additionally include cardiac electrical characteristics such as local activation time (LAT) of each vertex.

According to another embodiment of the present invention, one triangle selected among a plurality of triangles may include a first vertex, a second vertex, and a third vertex. In this specification, the first vertex may be denoted by i, the second vertex may be denoted by j, the third vertex may be denoted by k, or the first vertex may be denoted by p, the second vertex may be denoted by q, and the third vertex may be denoted by r. However, it is not limited thereto.

The term “local activation time” used herein may refer to a time at which an electrical signal has reached a specific point on the heart surface (e.g., a target vertex of a target triangle).” In this case, the local activation time may be key data showing how the electrical activation of the heart spatially spreads.

The term “mesh” used herein refers to a 3D network composed of a plurality of triangles for modeling a heart surface, and may reflect structural characteristics and conduction characteristics of the heart.

According to a feature of the present invention, the mesh may be composed of an isotropic structure or an anisotropic structure.

Meanwhile, according to the method for calculating the cardiac conduction velocity based on the conventional trigonometry, there may be a limitation in calculating the cardiac conduction velocity for a specific triangle in an anisotropic mesh structure.

In this case, “the cardiac conduction velocity” may mean a velocity at which an electrical signal of the heart is conducted through heart tissue.

In various embodiments, the cardiac conduction velocity may be calculated based on heart 3D data.

More specifically, in a 3D structure with respect to a heart, based on vertex coordinates of a target triangle selected among a plurality of triangles constituting the heart, a normal vector for the target triangle is calculated, and a cardiac conduction velocity for the target triangle may be determined based on the normal vector.

In this case, the term “normal vector (Nₜ)” used in the present invention refers to a unit vector perpendicular to the plane of the target triangle, and is calculated based on the vertex coordinate of the target triangle.

For example, the normal vector Nₜ for the target triangle may be determined by the product of the vectors (Vᵢ−V_{K}) and (Vₖ−Vᵢ) of two sides of the target triangle having the first to third vertices of i, j and k.

According to a feature of the present invention, a gradient may be calculated based on a normal vector and a local activation time value, and a cardiac conduction velocity to a target triangle may be determined based on the gradient.

The term “gradient” used herein represents a spatial variation in local activation time in a target triangle and may be calculated based on a normal vector, a difference in local activation time between vertices, and the area of the target triangle.

For example, the gradient may be calculated by normalizing the difference in local activation time between the two vertices of the target triangle to the normal vector and the area of the target triangle, and the specific calculation process will be described later.

The term “first gradient” used herein may be based on a difference in local activation time between the first vertex and the second vertex which form a target triangle and refer to a gradient calculated by reflecting the spatial relationship between a vector connecting the first vertex and the third vertex and a normal vector.

More specifically, the first gradient is calculated by combining a spatial characteristic with a change in the local activation time of the target triangle, and may be calculated by the following Equation 1.

$$First gradient = \frac{( {LAT}_{i} - {LAT}_{j} ) * {N}_{t} \times ( {v}_{i} - {v}_{k} )}{2 {A}_{t}} \qquad [ Equation 1 ]$$

Here, LATᵢ−LATₖ denotes a difference in local activation time between the first vertex and the second vertex, Nₜ denotes a normal vector, Vᵢ−Vₖ denotes a difference in position vector between the first vertex and the third vertex, and Aₜ denotes an area of a target triangle.

The term “second gradient” used herein may be based on the difference in local activation time between the third vertex and the first vertex of the target triangle and refer to a gradient calculated by reflecting the spatial relationship between the vector connecting the second vertex and the first vertex and the normal vector.

The second gradient is also calculated based on the change in local activation time and spatial characteristics, and may be calculated by Equation 2 below.

$$Second gradient = \frac{( {LAT}_{k} - {LAT}_{i} ) * {N}_{t} \times ( {v}_{j} - {v}_{i} )}{2 {A}_{t}} \qquad [ Equation 2 ]$$

Here, LATᵢ−LATⱼ means a difference in local activation time between the third vertex and the second vertex, Nₜ means a normal vector of the target triangle t, Vⱼ−Vᵢ means a difference in position vector between the second vertex and the first vertex, and Aₜ means the area of the target triangle.

Meanwhile, the area Aᵣ of the target triangle may be calculated by the Heron formula of Equation 3 below.

$$s = \frac{‖ {v}_{i} - {v}_{j} ‖ + ‖ {v}_{i} - {v}_{j} ‖ + ‖ {v}_{i} - {v}_{j} ‖}{2} , \qquad [ Equation 3 ]$$

$${A}_{t} = \sqrt{s ( s - ‖ {v}_{i} - {v}_{j} ‖ ) ( s - ‖ {v}_{i} - {v}_{k} ‖ ) ( s - ‖ {v}_{j} - {v}_{k} ‖ )}$$

Finally, ∇u(t), which is the gradient for the target triangle t, may be calculated by the sum of the first gradient and the second gradient as shown in Equation 4.

$$∇ u ( t ) = ( {LAT}_{i} - {LAT}_{j} ) \frac{{N}_{t} \times ( vi - vk )}{2 {A}_{t}} + ( {LAT}_{k} - {LAT}_{i} ) \frac{{N}_{t} \times ( {v}_{j} - {v}_{i} )}{2 {A}_{t}} \qquad [ Equation 4 ]$$

That is, according to the calculation method based on the first gradient and the second gradient, when any one surface of the triangle in the plurality of triangles is parallel with respect to the local activation time axis, that is, when the difference in the local activation time value is 0, the gradient can be calculated based on geometric characteristics such as a normal vector, the area of the triangle, and the difference in vector, so that the cardiac conduction velocity can be estimated.

According to another feature of the present invention, the gradient ∇ut for the target triangle may be in a reciprocal relationship with the cardiac conduction velocity CVₜ.

Accordingly, the CVₜ for the target triangle may be estimated by taking a reciprocal of the gradient for the target triangle.

According to various features of the present invention, a cardiac conduction velocity for a target vertex selected among vertices of a target triangle may be determined.

At this time, the cardiac conduction velocity for a selected target vertex can be obtained by calculating a gradient for each of the adjacent triangles including the target vertex as a vertex, determining a cardiac conduction velocity for each of the adjacent triangles based on the gradient, and calculating an average value of the cardiac conduction velocity for each of these.

For example, the cardiac conduction velocity CVₚ for the target vertex p may be calculated by Equation 5 below.

$${CV}_{p} = \frac{1}{{V}_{B ( p )}} {\int }_{B ( p )} {CV}_{t} dV \qquad [ Equation 5 ]$$

Here, B(p) is a cluster of adjacent triangles which are triangles defined as the adjacent region of the point p, V_{B(p)} is the volume or area of the region B(p), and CVₜ is the cardiac conduction velocity of each triangle belonging to B(p).

That is, the cardiac conduction velocity of the target vertex p may be calculated based on the average value of the cardiac conduction velocity of adjacent triangles.

According to the above calculation method, when one side of the triangle is parallel to the local activation time axis in a plurality of triangles constituting the heart structure, a gradient can be calculated based on the geometric characteristics such as a normal vector, the area of the triangle, and the difference in the vector, so that the cardiac conduction velocity can be estimated.

In particular, according to the above calculation method, the method for determining the cardiac conduction velocity according to various embodiments of the present invention may estimate a more accurate conduction velocity while preserving the phase of 3D data.

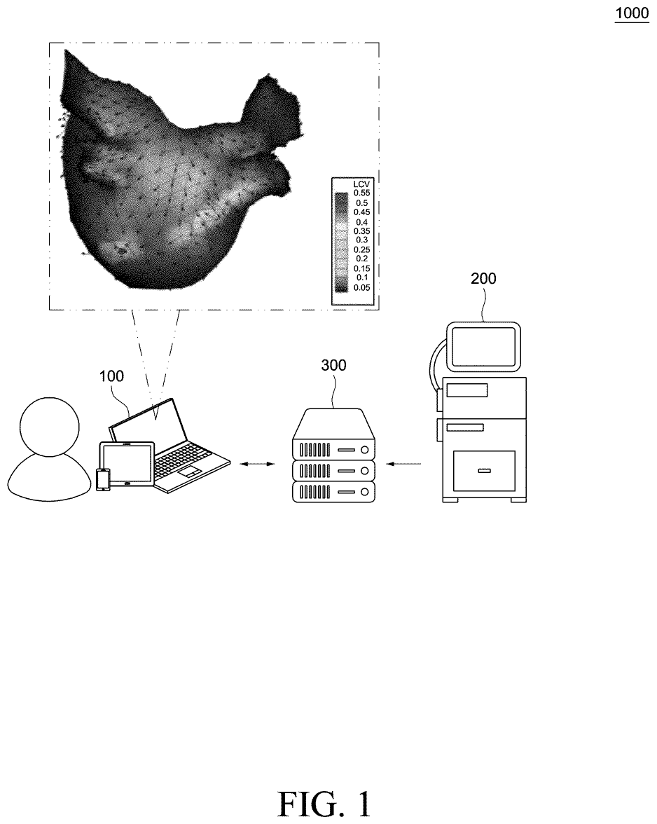

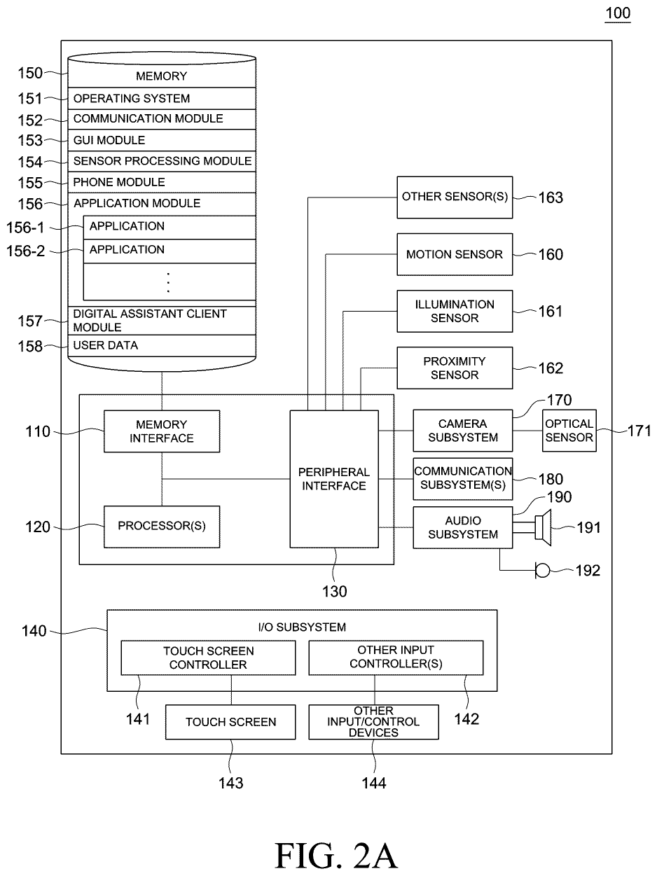

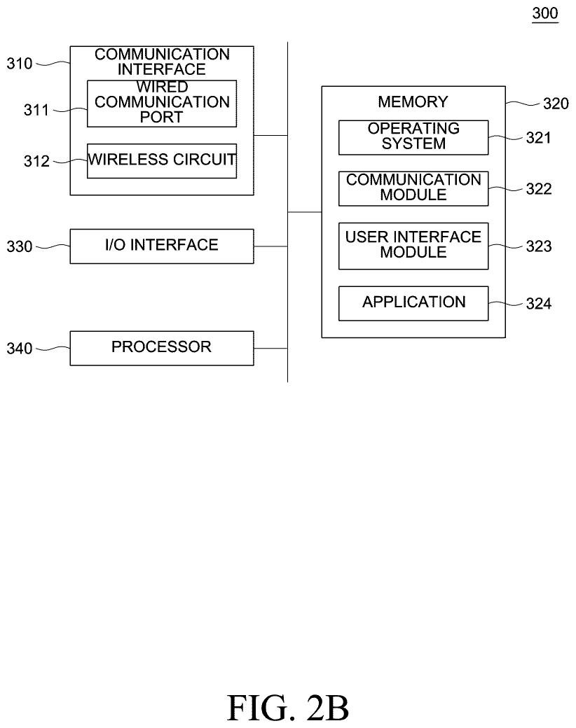

Hereinafter, a system for determining a cardiac conduction velocity using a device for determining a cardiac conduction velocity and a device for determining a cardiac conduction velocity will be described with reference to FIGS. 1, 2A, and 2B according to an embodiment of the present invention.

FIG. 1 illustrates a system for determining a cardiac conduction velocity using a device for determining a cardiac conduction velocity according to an embodiment of the present invention. FIG. 2A illustrates an exemplary configuration of a user device that is provided with information on a cardiac conduction velocity according to an embodiment of the present invention. FIG. 2B illustrates an exemplary configuration of a server for determining a cardiac conduction velocity according to an embodiment of the present invention.

First, referring to FIG. 1, the information providing system 1000 may be a system configured to provide a cardiac conduction velocity based on heart 3D data. In this case, the information providing system 1000 may be composed of a user device 100 that receives a cardiac conduction velocity, a cardiac 3D structure imaging device 200 that provides 3D data about a heart, and a decision server 300 that generates a cardiac conduction velocity based on the heart 3D data.

In various embodiments of the present invention, the user device 100 is an electronic device that provides a user interface for indicating a cardiac conduction velocity, and may include at least one of a smartphone, a tablet PC (Personal Computer), a laptop, and/or a PC.

The user device 100 may receive a cardiac conduction velocity of the determined object from the decision server 300 and may display it via a display unit (not shown).

The decision server 300 may include a general purpose computer, a laptop, and/or a data server that performs various operations for determining information related to the cardiac conduction velocity based on the heart 3D data. In this case, the decision server 300 may be a device for accessing a web server providing a web page or a mobile web server providing a mobile web site, but is not limited thereto.

In various embodiments, the decision server 300 may perform an operation to determine the cardiac conduction velocity based on the heart 3D data received from the cardiac 3D structure imaging device 200.

The decision server 300 may provide a cardiac conduction velocity to the user device 100.

The information provided from the decision server 300 may be provided as a web page through a web browser installed in the user device 100, or may be provided in the form of an application or a program. In various embodiments, such data may be provided in a form included in a platform in a client-server environment.

In more various embodiments, the cardiac 3D structure imaging device 200 comprises a imaging unit configured to capture a cardiac 3D structure and to generate heart 3D data including vertex coordinates for a plurality of triangles constituting the heart structure, a communication unit functionally connected to the imaging unit and configured to receive heart 3D data, and a processor functionally connected to the communication unit or the imaging unit.

In this case, the processor may be configured to calculate a normal vector for the target triangle based on vertex coordinates of a target triangle selected among the plurality of triangles, determine a cardiac conduction velocity for the target triangle based on the normal vector, and determine a cardiac conduction velocity for the target vertex based on the cardiac conduction velocity for an adjacent triangle including the target vertex selected among the vertices of the target triangle among the plurality of triangles.

Next, components of the user device 100 and the decision server 300 of the present invention will be described in detail with reference to FIGS. 2A and 2B.

First, referring to FIG. 2A, the user device 100 may include a memory interface 110, one or more processors 120, and a peripheral interface 130. Various components within user device 100 may be connected by one or more communication buses or signal lines.

The memory interface 110 may be connected to the memory 150 to transmit various data to the processor 120. Here, the memory 150 may include at least one type of storage medium among flash memory type, hard disk type, multimedia card micro type, card type memory (for example, SD or XD memory, etc.), RAM, SRAM, ROM, EEPROM, PROM, network storage, cloud, and blockchain data.

In various embodiments, the memory 150 may store at least one of an operating system 151, a communication module 152, a graphical user interface module (GUI) 153, a sensor processing module 154, a telephone module 155, and an application module 156. Specifically, the operating system 151 may include instructions for processing a basic system service and instructions for performing hardware tasks. Communication module 152 may communicate with at least one of other devices, computers, and servers. The graphic user interface module GUI 153 may process the graphic user interface. The sensor processing module 154 may process a sensor-related function (e.g., processing a received voice input using one or more microphones 192). The telephone module 155 may process a phone-related function. The application module 156 may perform various functions of a user application, such as electronic messaging, web browsing, media processing, browsing, imaging, and other process functions. In addition, the user device 100 may store one or more software applications 156-1, 156-2 (e.g., an information providing application) associated with any one type of service in the memory 150.

In various embodiments, the memory 150 may store a digital assistant client module 157 (hereinafter, referred to as a DA client module), and accordingly store instructions for performing functions on the client side of the digital assistant and various user data 158.

Meanwhile, the DA client module 157 may obtain a user's voice input, text input, touch input, and/or gesture input through various user interfaces (e.g., I/O subsystem 140) provided in the user device 100.

In addition, the DA client module 157 may output audio-visual and tactile types of data. For example, the DA client module 157 may output data composed of a combination of at least two of voice, sound, notification, text message, menu, graphic, video, animation, and vibration. In addition, the DA client module 157 may communicate with a digital assistant server (not shown) using the communication subsystem 180.

In various embodiments, the DA client module 157 may collect additional information about the surrounding environment of the user device 100 from various sensors, subsystems, and peripheral devices to configure a context related to user input. For example, the DA client module 157 may provide context information to a digital assistant server along with the user input to infer the user's intent. Here, the context information that may be accompanied by a user input may include sensor information, for example, lighting, ambient noise, ambient temperature, an image of the surrounding environment, video, and the like. For another example, the context information may include a physical state of the user device 100 (e.g., device orientation, device location, device temperature, power level, speed, acceleration, motion pattern, cellular signal strength, etc.). As another example, the context information may include information related to a software state of the user device 100 (e.g., a process running on the user device 100, an installed program, past and current network activity, background service, error log, resource use, etc.).

In various embodiments, the memory 150 may include additional or deleted instructions, and further, the user device 100 may include additional configurations in addition to the configurations illustrated in FIG. 2A, or may exclude some configurations.

Processor 120 may control the overall operation of user device 100 and may execute various instructions for implementing an interface that provides a cardiac conduction velocity by running an application or program stored in memory 150.

The processor 120 may correspond to a computing device such as a central processing unit (CPU) or an application processor (AP). In addition, the processor 120 may be implemented in the form of an integrated chip (IC) such as a System on Chip (SoC) in which various computing devices such as a Neural Processing Unit (NPU) are integrated.

Peripheral interface 130 may be connected to various sensors, subsystems, and peripheral devices to provide data so that user device 100 may perform various functions. Here, it may be understood that function performed by the user device 100 is performed by the processor 120.

The peripheral interface 130 may receive data from the motion sensor 160, the illumination sensor (optical sensor) 161, and the proximity sensor 162, and through this, the user device 100 may perform orientation, lighting, and proximity sensing functions. As another example, peripheral interface 130 may receive data from other sensors 163 (positioning system-GPS receiver, temperature sensor, biometric sensor), thereby allowing user device 100 to perform functions related to other sensors 163.

In various embodiments, the user device 100 may include a camera subsystem 170 connected to the peripheral interface 130 and an optical sensor 171 connected thereto, through this the user device 100 may perform various imaging functions such as photographing and video clip recording.

In various embodiments, user device 100 may include communication subsystem 180 connected to peripheral interface 130. Communication subsystem 180 consists of one or more wired/wireless networks and may include various communication ports, radio frequency transceivers, and optical transceivers.

In various embodiments, the user device 100 comprises an audio subsystem 190 connected with a peripheral interface 130, such an audio subsystem 190 comprising one or more speakers 191 and one or more microphones 192, such that the user device 100 may perform voice-activated functions, such as voice recognition, voice replication, digital recording, and telephone functions.

In various embodiments, user device 100 may include I/O subsystem 140 connected with peripheral interface 130. For example, the I/O subsystem 140 may control the touch screen 143 included in the user device 100 via the touch screen controller 141. As an example, the touch screen controller 141 may detect user's contact and movement or cessation of user's contact and movement using any one of a plurality of touch sensing technologies, such as capacitive, resistive, infrared, surface acoustic wave technology, proximity sensor array, etc. As another example, I/O subsystem 140 may control other input/control device 144 included in user device 100 via other input controller(s) 142. As an example, the other input controller(s) 142 may control pointer devices such as one or more buttons, a rocker switch, a thumb wheel, an infrared port, a USB port, and a stylus.

Next, referring to FIG. 2B, the decision server 300 may include a communication interface 310, a memory 320, an I/O interface 330, and a processor 340, and each component may communicate with each other via one or more communication buses or signal lines.

The communication interface 310 may be connected to the user device 100 and the cardiac 3D structure imaging device 200 via a wired/wireless communication network to exchange data. For example, the communication interface 310 may receive values corresponding to the heart 3D data from the cardiac 3D structure imaging device 200 or the user device 100, and determine a cardiac conduction velocity therefrom and transmit the same to the user device 100.

Meanwhile, a communication interface 310 enabling transmission and reception of such data includes a wired communication port 311 and a wireless circuit 312, wherein the wired communication port 311 may include one or more wired interfaces, for example, Ethernet, a universal serial bus (USB), a Firewire, and the like. In addition, the wireless circuit 312 may transmit and receive data to and from an external device through an RF signal or an optical signal. In addition, wireless communication may use at least one of a plurality of communication standards, protocols and technologies, such as GSM, EDGE, CDMA, TDMA, Bluetooth, Wi-Fi, VOIP, Wi-MAX, or any other suitable communication protocol.

The memory 320 may store various data used in the decision server 300. For example, the memory 320 may store more various pieces of heart 3D data, or may store a mathematical calculation model for performing prediction on a cardiac conduction velocity.

In various embodiments, memory 320 may include a volatile or nonvolatile recording medium capable of storing various data, commands, and information. For example, the memory 320 may include at least one type of storage medium among flash memory type, hard disk type, multimedia card micro type, card type memory (for example, SD or XD memory, etc.), RAM, SRAM, ROM, EEPROM, PROM, network storage, cloud, and blockchain data.

In various embodiments, the memory 320 may store the configuration of at least one of the operating system 321, the communication module 322, the user interface module 323, and one or more applications 324.

Operating system 321 (e.g., a built-in operating system such as LINUX, UNIX, MAC OS, WINDOWS, VxWorks, etc.) may include various software components and drivers for controlling and managing general system tasks (e.g., memory management, storage device control, power management, etc.), and may support communication between various hardware, firmware, and software components.

The communication module 323 may support communication with other devices through the communication interface 310. The communication module 320 may include various software components for processing data received by the wired communication port 311 of the communication interface 310 or the wireless circuit 312.

The user interface module 323 may receive a user's request or input from a keyboard, a touch screen, a microphone, or the like through the I/O interface 330, and may provide a user interface on a display.

Application 324 may include a program or module configured to be executed by one or more processors 340. Here, an application for providing a cardiac conduction velocity may be implemented on a server farm.

The I/O interface 330 may connect at least one of an input/output device (not shown) of the decision server 300, for example, a display, a keyboard, a touch screen, and a microphone, to the user interface module 323. The I/O interface 330 may receive a user input (e.g., voice input, keyboard input, touch input, etc.) together with the user interface module 323 and process a command according to the received input.

The processor 340 may be connected to the communication interface 310, the memory 320, and the I/O interface 330 to control the overall operation of the decision server 300 and perform various commands for providing information through an application or a program stored in the memory 320.

The processor 340 may correspond to a computing device such as a central processing unit (CPU) or an application processor (AP). In addition, the processor 340 may be implemented in the form of an integrated chip (IC) such as a system on chip (SoC) in which various computing devices are integrated. Alternatively, the processor 340 may include a module for calculating an artificial neural network model such as a neural processing unit (NPU).

In various embodiments, processor 340 may be configured to determine that the risk of developing a heart disease is high when the cardiac conduction velocity is below or above a predetermined level.

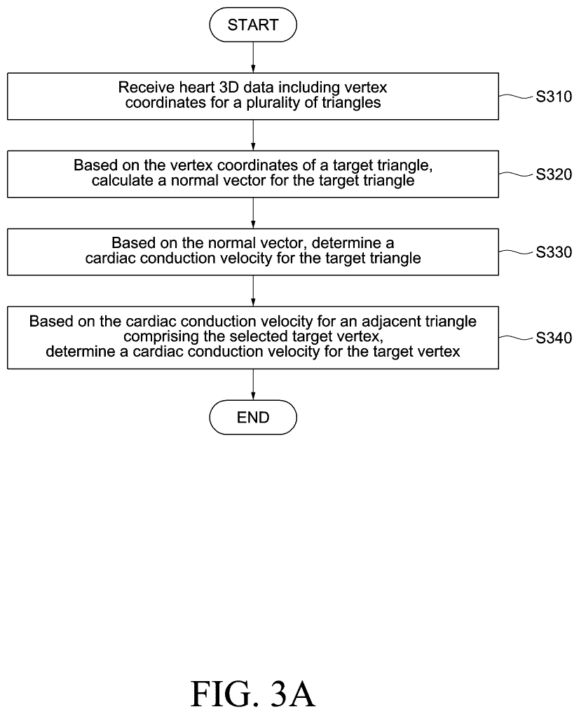

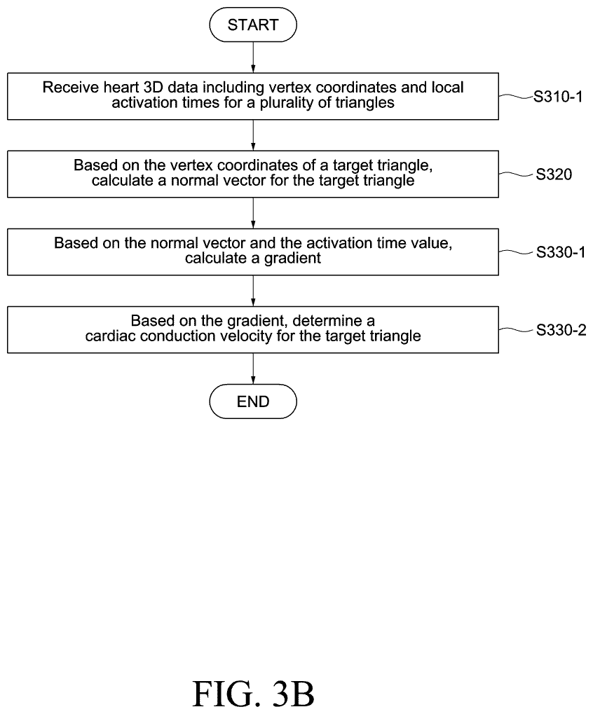

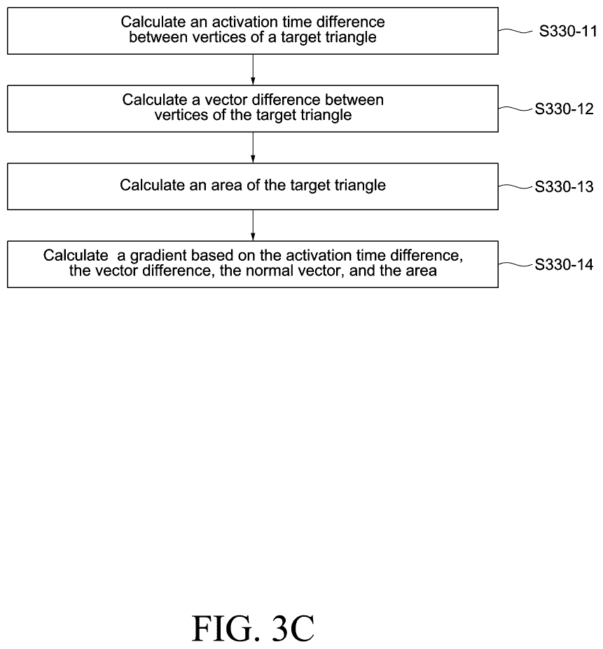

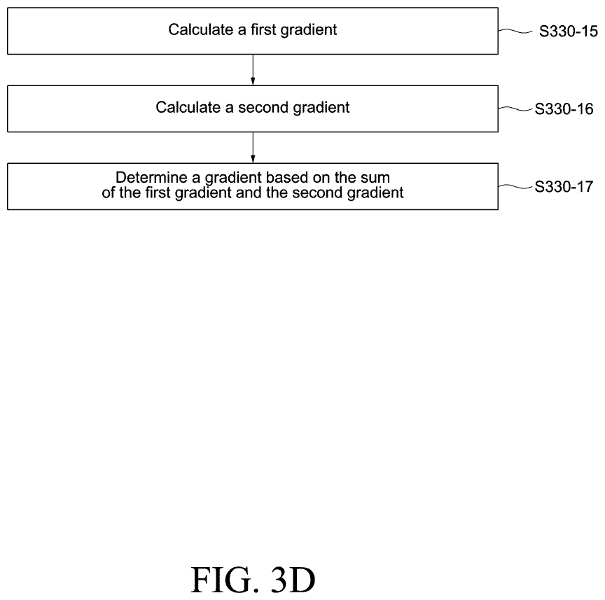

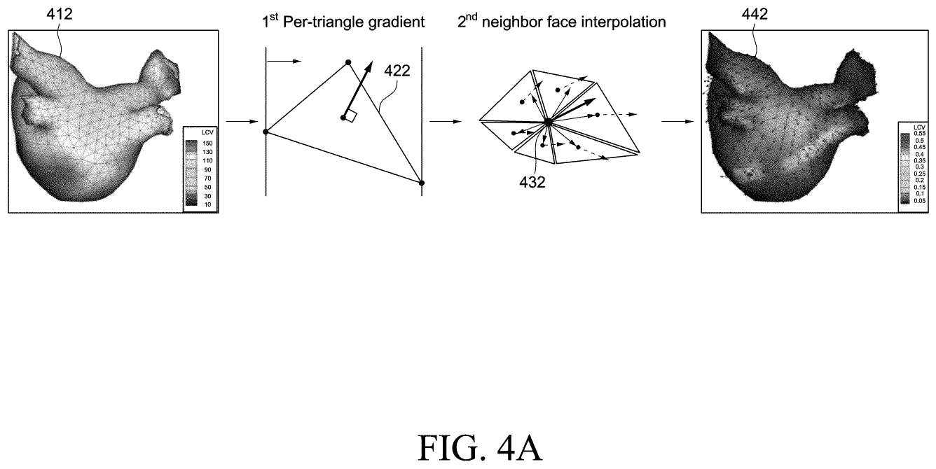

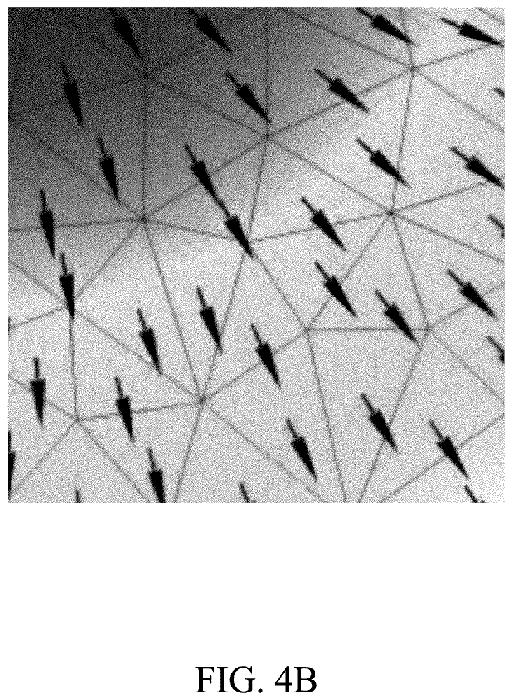

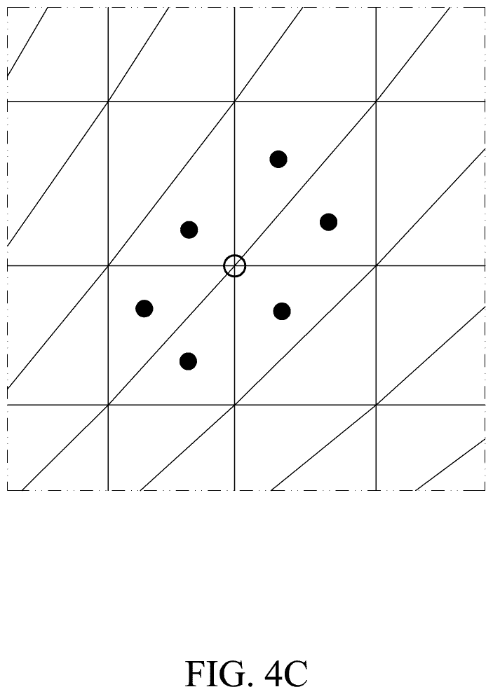

Hereinafter, a method for determining a cardiac conduction velocity according to an embodiment of the present invention will be described in detail with reference to FIGS. 3A to 3D and 4A to 4C.

FIGS. 3A to 3D illustrate a procedure of a method for determining a cardiac conduction velocity according to an embodiment of the present invention. FIGS. 4A to 4C exemplarily illustrate a process of a method for determining a cardiac conduction velocity according to an embodiment of the present invention.

In this case, it may be interpreted that the process of determining the cardiac conduction velocity to be described later is performed through the above-described decision server 300 or the processor (not shown) of the cardiac 3D structure imaging device 200.

First, referring to FIG. 3A, in order to determine a cardiac conduction velocity, heart 3D data including vertex coordinates for a plurality of triangles constituting a heart structure is received (S310), and based on the vertex coordinates of a target triangle selected among a plurality of triangles, a normal vector for the target triangle is calculated (S320). Then, a cardiac conduction velocity for the target triangle is determined based on the normal vector (S330), and a cardiac conduction velocity for the target vertex is determined based on the cardiac conduction velocity for an adjacent triangle comprising a target vertex selected among the vertices of the target triangle (S340).

According to a feature of the present invention, in step (S310) in which heart 3D data is received, the plurality of triangles may be configured in a mesh structure corresponding to electrical signal conduction of the heart with respect to the heart surface.

According to another feature of the present invention, the plurality of triangles may be configured in an isotropic mesh structure or an anisotropic mesh structure according to conduction characteristics of heart tissue.

Referring to FIG. 3B, according to various embodiments of the present invention, heart 3D data including vertex coordinates and local activation times for a plurality of triangles constituting a heart structure is received (S310-1), a normal vector is calculated (S320), then a gradient is calculated based on the normal vector and the activation time value (S330-1), and a cardiac conduction velocity for a target triangle is determined based on the gradient (S330-2).

In this case, in the step (S330-2) of estimating the cardiac conduction velocity with respect to the target triangle, the cardiac conduction velocity for each of the adjacent triangles is determined based on the gradient, and an average value of the cardiac conduction velocity for each is calculated, so that the cardiac conduction velocity with respect to the target vertex may be determined.

Further referring to FIG. 3C, according to various embodiments of the present invention, an activation time difference between vertices of a target triangle is calculated (S330-11), a vector difference between vertices of the target triangle is calculated (S330-12), an area of the target triangle is calculated (S330-13), and a gradient may be estimated based on the activation time difference, the vector difference, the normal vector, and the area (S330-14).

For example, referring to FIGS. 4A and 4B together, heart 3D data 412 including vertex coordinates and local activation times for a plurality of triangles constituting a heart structure is received, and then a normal vector for each of the plurality of triangles including a target triangle 422 is determined. Then, the gradient is calculated based on the vector difference, the normal vector and the activation time difference, and the area.

In this case, ∇u(t), which is the gradient for the target triangle t, may be determined by based on Equation 4.

$$∇ u ( t ) = ( {LAT}_{i} - {LAT}_{j} ) \frac{{N}_{t} \times ( vi - vk )}{2 {A}_{t}} + ( {LAT}_{k} - {LAT}_{i} ) \frac{{N}_{t} \times ( {v}_{j} - {v}_{i} )}{2 {A}_{t}} \qquad [ Equation 4 ]$$

Here, LATᵢ−LATⱼ and LATᵢ−LATⱼ denote a difference in local activation time between the vertices respectively, Nr denotes a normal vector for the target triangle, Vᵢ−Vₖ and Vⱼ−Vᵢ denotes a difference in position vector between the vertices respectively, and Aᵣ denotes an area of a target triangle.

Meanwhile, the gradient ∇ut for the target triangle may be in a reciprocal relationship with the cardiac conduction velocity CVₜ.

Accordingly, the CVₜ for the target triangle may be estimated by taking a reciprocal of the gradient for the target triangle.

According to various features of the present invention, a cardiac conduction velocity for a target vertex selected among vertices of a target triangle may be determined.

More specifically, referring to FIGS. 4A and 4C together, the cardiac conduction velocity for each adjacent triangle including the target vertex 432 is determined, and the average value of the cardiac conduction velocity for each is calculated, thereby determining a cardiac conduction velocity for the target vertex.

At this time, the cardiac conduction velocity CVₚ for the target vertex p may be calculated by Equation 5 below.

$${CV}_{p} = \frac{1}{{V}_{B ( p )}} {\int }_{B ( p )} {CV}_{t} dV \qquad [ Equation 5 ]$$

Here, B(p) is a cluster of adjacent triangles which are triangles defined as the adjacent region of the point p, V_{B(p)} is the volume or area of the region B(p), and CVₜ is the cardiac conduction velocity of each triangle belonging to B(p).

That is, the cardiac conduction velocity of the target vertex p may be calculated based on the average value of the cardiac conduction velocity of adjacent triangles.

Therefore, based on the normal vector and the gradient according to the local activation time of the target triangle, it may be possible to estimate the cardiac conduction velocity from the cardiac data of the 3D mesh structure. This calculation method is not limited to the target triangle and the direction of the local activation time, and may be a calculation method capable of estimating the cardiac conduction velocity in any situations.

In particular, in a plurality of triangles constituting the heart structure, when one side of the triangle is parallel to the local activation time axis, a gradient can be calculated based on geometric characteristics such as a normal vector, the area of the triangle, and the difference in the vector, so that the cardiac conduction velocity can be estimated.

On the other hand, the calculation of the gradient for the target triangle is not limited to the method described above.

Referring to FIG. 3D, in more various embodiments, the target triangle includes a first vertex, a second vertex, and a third vertex, a first gradient is calculated, which is defined as a gradient based on the normal vector and the difference in activation time between the first and second vertices (S330-15), and a second gradient is calculated, which is defined as a gradient based on the normal vector and the difference in activation time between the second and third vertices (S330-16), and the gradient of the target triangle may be determined based on the sum of the first gradient and the second gradient (S330-17).

More specifically, in the step S330-15 where the first gradient is calculated, the first gradient is calculated by combining the spatial characteristics with the change in the local activation time of the target triangle, and may be calculated by the following Equation 1.

$$First gradient = \frac{( {LAT}_{i} - {LAT}_{j} ) * {N}_{t} \times ( {v}_{i} - {v}_{k} )}{2 {A}_{t}} \qquad [ Equation 1 ]$$

Here, LATᵢ−LATⱼ denotes a difference in local activation time between the first vertex and the second vertex, Nₜ denotes a normal vector, Vᵢ−Vₖ denotes a difference in position vector between the first vertex and the third vertex, and Aₜ denotes an area of a target triangle.

Then, in step S330-16 in which the second gradient is calculated, the second gradient is also calculated based on the change of the local activation time and the spatial characteristics, and may be calculated by Equation 2 below.

$$Second gradient = \frac{( {LAT}_{k} - {LAT}_{i} ) * {N}_{t} \times ( {v}_{j} - {v}_{i} )}{2 {A}_{t}} \qquad [ Equation 2 ]$$

Here, LATₖ−LATᵢ means a difference in local activation time between the third vertex and the second vertex, Nₜ means a normal vector of the target triangle t, Vⱼ−Vᵢ means a difference in position vector between the second vertex and the first vertex, and Aₜ means the area of the target triangle.

Meanwhile, the area Aₜ of the target triangle may be calculated by the Heron formula of Equation 3 below.

$$s = \frac{‖ {v}_{i} - {v}_{j} ‖ + ‖ {v}_{i} - {v}_{j} ‖ + ‖ {v}_{i} - {v}_{j} ‖}{2} , \qquad [ Equation 3 ]$$

$${A}_{t} = \sqrt{s ( s - ‖ {v}_{i} - {v}_{j} ‖ ) ( s - ‖ {v}_{i} - {v}_{k} ‖ ) ( s - ‖ {v}_{j} - {v}_{k} ‖ )}$$

Finally, ∇u(t), which is the gradient for the target triangle t, may be calculated by the sum of the first gradient and the second gradient as shown in Equation 4.

$$∇ u ( t ) = ( {LAT}_{i} - {LAT}_{j} ) \frac{{N}_{t} \times ( vi - vk )}{2 {A}_{t}} + ( {LAT}_{k} - {LAT}_{i} ) \frac{{N}_{t} \times ( {v}_{j} - {v}_{i} )}{2 {A}_{t}} \qquad [ Equation 4 ]$$

That is, according to the calculation method based on the first gradient and the second gradient, when any one surface of the triangle in the plurality of triangles is parallel with respect to the local activation time axis, that is, when the difference in the local activation time value is 0, the gradient can be calculated based on geometric characteristics such as a normal vector, the area of the triangle, and the difference in vector, so that the cardiac conduction velocity can be estimated.

Accordingly, in the method for determining the cardiac conduction velocity according to various embodiments of the present invention, a more accurate conduction velocity may be estimated while preserving the topology of the 3D data.

In particular, by providing a system capable of stably calculating a cardiac conduction velocity even in a 3D mesh environment in which the size, ratio, arrangement, etc. of triangles are irregular or uneven, it is possible to overcome the limitations of the prior art and produce results suitable for various clinical environments.

Furthermore, the method for determining the cardiac conduction velocity, according to various embodiments of the present invention, can be applied to heart 3D data with various anatomical conditions and physical properties by providing stable and consistent results even in complex structural environments including both anisotropy and isotropy.

Returning to FIG. 3A, in more various embodiments of the present invention, after the step S340 in which the cardiac conduction velocity is determined, when the cardiac conduction velocity is below or above a predetermined level, the determining that the risk of developing heart disease is high may be further performed.

Accordingly, the method for determining the cardiac conduction velocity according to various embodiments of the present invention provides a system capable of quantitatively analyzing the transmission path of electrical signals in the heart, thereby contributing to accurate diagnosis and treatment planning of cardiovascular diseases including arrhythmia.

### Assessment: Estimation of Cardiac Conduction Velocity Based on Heart 3D Data

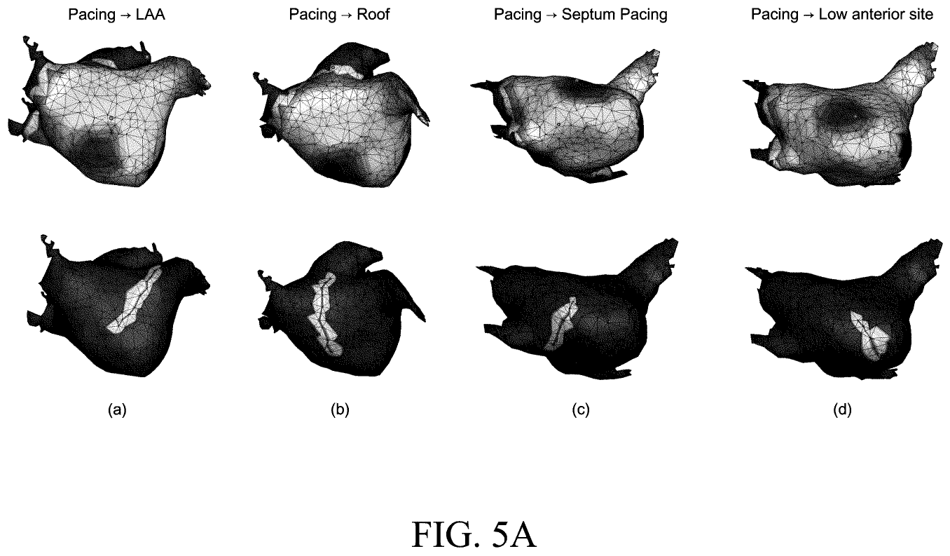

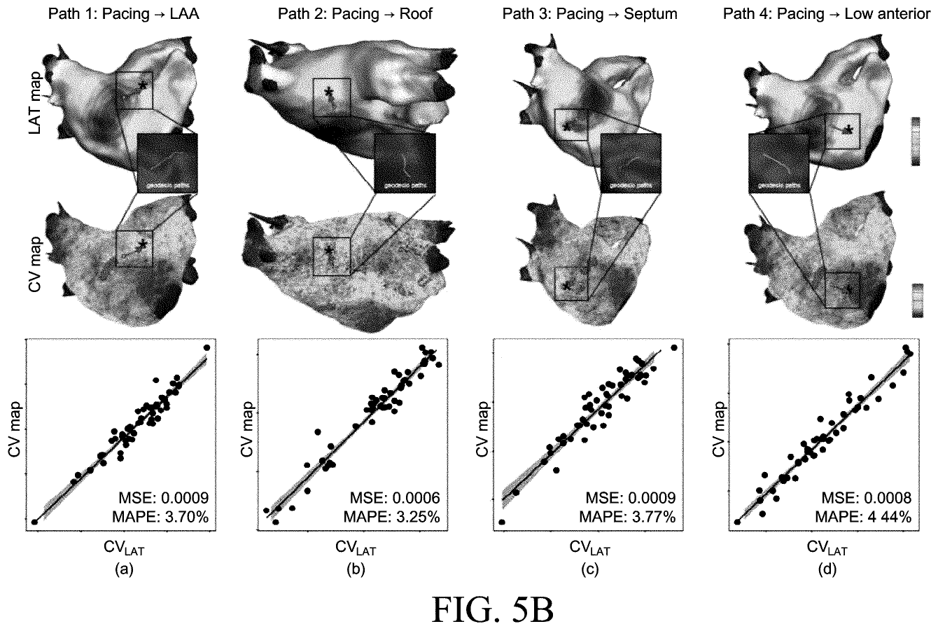

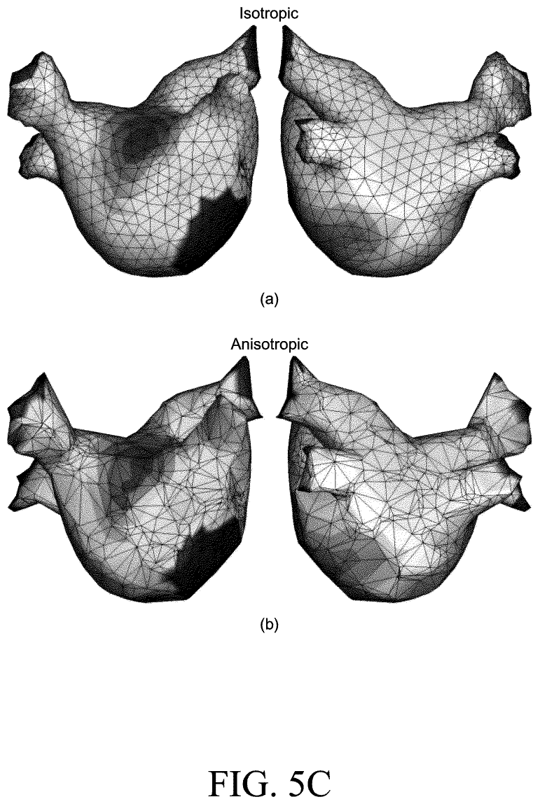

Hereinafter, an assessment result of a method for determining a cardiac conduction velocity according to various embodiments of the present invention will be described with reference to FIGS. 5A to 5C.

First, referring to FIG. 5A, for this assessment, in four parts of the heart (Pacing→Left Atrial Appendage (LLA), Pacing→Roof, Pacing→Septum, Pacing→Low anterior site), the gradient-based electrical conduction velocity calculation method according to various embodiments of the present invention was compared with the electrical conduction velocity (geodetic velocity, v) calculated based on the curve distance s measured on the 3D surface of the heart and the accumulated integration activation time t.

More specifically, referring to (a), (b), (c), and (d) of FIG. 5B together, the results of comparing the electrical conduction velocity (CV_{LAT}) calculated based on local activation time according to various embodiments of the present invention for determining the cardiac conduction velocity of four parts of the heart and the conduction velocity (CV_{gradient}) calculated based on the geodetic path-based curve distance s and the accumulated integration activation time t are shown.

At this time, statistical analysis shows that both calculation methods have little error. This may mean a result of proving the effectiveness of the method for determining the conduction velocity according to various embodiments of the present invention.

Further referring to FIG. 5C, in a mesh generated by a conventional trigonometry-based cardiac conduction velocity calculation method, it was difficult to calculate cardiac conduction velocity for about 3% of triangles, but the method for determining cardiac conduction velocity according to various embodiments of the present invention can be applied to both isotropic and anisotropic meshes, thereby reducing the proportion of triangles that cannot be calculated.

That is, the method for determining the cardiac conduction velocity according to various embodiments of the present invention can accurately estimate conduction velocity regardless of the spatial resolution and uniformity of the triangle, and can be used to accurately estimate the conduction velocity in clinical practice based on a high-resolution cardiac mapping system.

Accordingly, the present invention provides a system capable of stably calculating a cardiac conduction velocity even in a 3D mesh environment in which the size, ratio, and arrangement of triangles are irregular or uneven, so that it is possible to overcome the limitations of the prior art and derive results suitable for various clinical environments.

More specifically, the present invention can be applied to heart 3D data with various anatomical conditions and physical properties by providing stable and consistent results even in complex structural environments including both anisotropy and isotropy.

Furthermore, the present invention can contribute to the accurate diagnosis and treatment planning of cardiovascular diseases, including arrhythmia, by providing a system capable of quantitatively analyzing the transmission path of electrical signals in the heart.

That is, the present invention can provide highly accurate information on the cardiac conduction velocity, which is clinically important, thereby contributing to the development of personalized treatment strategies for patients with cardiovascular disease and the prediction of disease prognosis.

Although the embodiments of the present invention have been described in more detail with reference to the attached drawings, the present invention is not necessarily limited to these embodiments, and various modifications can be made without departing from the technical idea of the present invention. Accordingly, the embodiments disclosed in the present invention are not intended to limit the technical idea of the present invention, but to explain it, and the scope of the technical idea of the present invention is not limited by these embodiments. Therefore, it should be understood that the embodiments described above are exemplary in all aspects and not restrictive. The protection scope of the present invention should be interpreted by the claims below, and all technical ideas within the equivalent scope should be interpreted as being included in the scope of the rights of the present invention.

What is claimed is:

1. A method for determining a cardiac conduction velocity implemented by a processor, comprising:
receiving heart 3D data including vertex coordinates for a plurality of triangles constituting a heart structure;
calculating a normal vector for a target triangle based on the vertex coordinates of the target triangle selected among the plurality of triangles;
determining the cardiac conduction velocity for the target triangle based on the normal vector; and
determining the cardiac conduction velocity for the target vertex based on an cardiac conduction velocity for an adjacent triangle including the target vertex selected among the vertices of the target triangle among the plurality of triangles.

2. The method of claim 1,
the heart 3D data further comprises:
a local activation time (LAT) corresponding to each vertex coordinate; and
the determining the cardiac conduction velocity for the target triangle comprises:
calculating a gradient based on the normal vector and the local activation time value, and
determining the cardiac conduction velocity for the target triangle based on the gradient.

3. The method of claim 2,
the calculating the gradient comprises:
calculating a difference in the local activation time between vertices of the target triangle;
calculating a difference in vector between vertices of the target triangle;
calculating an area of the target triangle; and
calculating the gradient based on the difference in the local activation time, the difference in vector, the normal vector, and the area.

4. The method of claim 3,
the target triangle includes a first vertex, a second vertex, and a third vertex, and
the calculating the gradient comprises:
calculating a first gradient;
calculating a second gradient; and
determining the gradient based on a sum of the first gradient and the second gradient; and
the first gradient is defined as:
a gradient based on the normal vector and a difference in the local activation time between the first vertex and the second vertex; and
the second gradient is be defined as:
a gradient based on the normal vector and a difference in the local activation time between the second vertex and the third vertex.

5. The method of claim 1,
the determining the cardiac conduction velocity comprises:
calculating a gradient for each of the adjacent triangles;
determining a cardiac conduction velocity for each of the adjacent triangles based on the gradient; and
calculating an average value of the cardiac conduction velocity for each of the adjacent triangles.

6. The method of claim 1,
the plurality of triangles is configured in:
a mesh structure corresponding to electrical signal conduction of the heart with respect to the heart surface.

7. The method of claim 6,
the plurality of triangles is configured in:
an isotropic or an anisotropic mesh structure according to conduction characteristics of heart tissue.

8. The method of claim 1, comprising:
after the determining the cardiac conduction velocity,
determining that if the cardiac conduction velocity is below or above a predetermined level, the risk of developing a heart disease is high.

9. A device for determining a cardiac conduction velocity, comprising:
a communication unit configured to receive heart 3D data comprising vertex coordinates for a plurality of triangles constituting a heart structure, and
a processor functionally connected with the communication unit,
wherein the processor is configured to:
calculate a normal vector for a target triangle based on the vertex coordinates of the target triangle selected among the plurality of triangles,
determine the cardiac conduction velocity for the target triangle based on the normal vector, and
determine the cardiac conduction velocity for the target vertex based on cardiac conduction velocity for an adjacent triangle including the target vertex selected among the vertices of the target triangle among the plurality of triangles.

10. The device of claim 9,
the heart 3D data comprises:
a local activation time (LAT) corresponding to each vertex coordinate, and
the processor is further configured to:
calculate a gradient based on the normal vector and the local activation time value, and
determine the cardiac conduction velocity for the target triangle based on the gradient.

11. The device of claim 10,
the processor is further configured to:
calculate a difference in the local activation time between vertices of the target triangle;
calculate a difference in vector between vertices of the target triangle;
calculate an area of the target triangle; and
calculate the gradient based on the difference in the local activation time, the difference in vector, the normal vector, and the area.

12. The device of claim 10,
the target triangle comprises a first vertex, a second vertex, and a third vertex; and
the processor is further configured to:
calculate the first gradient;
calculate the second gradient; and
determine the gradient based on a sum of the first gradient and the second gradient;
the first gradient being defined as:
a gradient based on the normal vector, and a difference in the local activation time between the first vertex and the second vertex; and
the second gradient being defined as:
a gradient based on the normal vector and a difference in the local activation time between the second vertex and the third vertex.

13. The device of claim 9,
the processor is further configured to:
calculate a gradient for each of the adjacent triangles,
determine a cardiac conduction velocity for each of the adjacent triangles based on the gradient, and
calculate an average value of the cardiac conduction velocity for each of the adjacent triangles.

14. The device of claim 9,
the plurality of triangles is configured in:
a mesh structure corresponding to electrical signal conduction of the heart with respect to the heart surface.

15. The device of claim 14,
the plurality of triangles is configured in:
an isotropic mesh or an anisotropic mesh structure according to conduction characteristics of heart tissue.

16. The device of claim 9,
the processor is further configured to:
determine that if the cardiac conduction velocity is below or above a predetermined level, the risk of developing a heart disease is high.

17. A cardiac 3D structure imaging device, comprising:
an imaging unit configured to capture a 3D structure of a heart, and generate heart 3D data consisting of a plurality of triangles and including the vertex coordinates of a plurality of triangles constituting the heart structure;
a communication unit functionally connected to the imaging unit and configured to receive heart 3D data; and
a processor functionally connected to the communication unit or the imaging unit; and
the processor is configured to:
calculate a normal vector for a target triangle based on vertex coordinates of the target triangle selected among the plurality of triangles;
determine a cardiac conduction velocity for the target triangle based on the normal vector; and
determine a cardiac conduction velocity for the target vertex based on a cardiac conduction velocity for an adjacent triangle including a target vertex selected among vertices of a target triangle among the plurality of triangles.

18. A system for determining a cardiac conduction velocity, comprising:
an internal memory configured to store heart 3D data including vertex coordinates for a plurality of triangles constituting a heart structure; and
the system is configured to:
calculate a normal vector for a target triangle based on the vertex coordinates of the target triangle selected among the plurality of triangles;
determine the cardiac conduction velocity for the target triangle based on the normal vector; and
determine the cardiac conduction velocity for the target vertex based on a cardiac conduction velocity for an adjacent triangle including a target vertex selected among vertices of the target triangle among the plurality of triangles.
